SEQUENCE LISTING (18 sequences)

SEQ ID NO: 1 - DNA, 370 bp, Rattus sp.
caagtcaagtttttttattttattgtcagttacatgctttatagaaaaaagtgtggagaaccggtcagggttgtacaaaaaaaggctaggttcctacgttgttttatttacaccattgtgaggacgcccccacttcaggcgcagcagctgcacttgtccgaagcctctttgcagatgcagccctgggagcacttcgcacagcccacggggcagcaggaacagcagcttttcttgcaggaggtgcatttgcattgtttgcatttgcaggagccagcgcaggagcaggatccatctgtggcacaggagcagttggggtccatggcgaatggaggcggcagttggagatcaacgagagatcgctcctcgtgcc

SEQ ID NO: 2 - DNA, 782 bp, Rattus norvegicus
caaccagagagtgagccgagacagcaaacatcagatcgtgccccgacccacgccagcatgggctccttctccatcaccaagggcttcttcctcttcctggccttttggctcccaggccatattggagcaaatcccgtatacagtgcggtgtccaacacagatctgatggatttcaagaacctgctagaccacctggaggagaagatgccggtagaagatgaggtcatgcctccgcaggccctgagcgagcagaccgatgaagcgggggcggcacttagctccctctctgaggtgcctccctggactggggaagtcaacccgtctcagagagatggaggtgctctcgggcgcggcccctgggacccctccgatagatctgccctcttgaaaagcaaactgagggctctgctcgctggccctcggagcctgcgaaggtcaagctgcttcgggggtaggattgacaggattggagcccagagcggactaggctgcaacagcttccggtaccgaagataacagccaaatctgctcgagcagatcgcaaaagatcccaagccttgcggtgtgtcacacagcttggtcgcattgccactgagaggtggtgaataccctcctggagctgcagcttcctgtcttcatctatcacgatcgatgttaagtgtagatgagtggtttagtgaggccttacctctcccactctgcatattaaggtagatcctcacccctttcagaaagcagtttggaaaaaaataaatccgaataaacttcagcaccacggacag

SEQ ID NO: 3 - DNA, 1907 bp, Rattus leucopus
ccacgcgtccgcgaacctactgaacactccaccggcggcggctcggttccccgccagccgcccagcgcgcagacggggcttgatgtccaggttgtcagcgcagctggcgactcaggtaccatgagccaggacaccgaagtggacatgaaagatgtggagctgaacgagctggaaccggagaagcagcctatgaatgcagcggacggggcggcagccggggagaagaacggtctggtgaagattaaggtggccgaagacgaggcggaagccggggtcaagttcacaggcttatccaaggaggagctattgaaggtagctggcagcccgggctgggtgcgcacccgctgggcgctgctgctgctcttctggctcggttggctgggtatgctggcgggcgccgtggttatcatcgttcgggcgccacgctgccgtgagctgccggtacagagatggtggcacaagggcgccctctaccgcatcggcgaccttcaggccttcgtaggcccggaagcgagaggcatagctggtctgaagaaccatctggagtacttgagcaccctgaaggtgaagggcctagttttgggcccaattcacaagaaccagaaggatgaagtcaatgaaaccgacttgaaacagattgatcccgatttaggctcccaggaagattttaaagaccttctacaaagtgccaagaaaaagagcattcacatcattttggacctcactcccaactataagggccagaatgcatggttcctccctcctcaggctgacattgtagccaccaaaatgaaggaggctctgagttcttggttgcaggacggtgtggatgggttccaagttcgggatgtgggaaagctggcgaatgcatccttgtacttggctgagtggcagaatatcaccaagaacttcagtgaggacaggcttttgattgcagggaccgcgtcctctgacctgcaacaaattgtcaacatacttgaatccaccagcgatctgctgctgaccagctcatacctgtcacagcccgttttcactggggagcatgcagaactcctagtgattaagtatttgaatgccactggcagccgctggtgcagctggagtgtgtcgcaggcaggactcctgacatcctttataccggctcagtttctccgactctaccagctgctgctcttcactctgccaggaactcctgttttcagctatggggatgagcttggccttcaggcagttgcccttcctggacagcctatggaggctccattcatgctgtggaatgagtctagcaactcccaaacctcaagtcctgtaagcctcaacatgacagtgaagggccaaaatgaagaccccggctccctcctcacccagttccggcgactgagtgacctccgtggtaaggagcgctctctgttacacggtgactttgatgcactgtcttcctcatctggcctcttctcctacgtccgccactgggaccagaatgagcgttacctggtggtgctcaacttccaggatgtgggcctgtcagccagggtaggagcctccaacctccctgctggcataagcctgccagccagtgctaaccttttgcttagtactgacagcacccggctaagccgtgaggagggcacctccctgagcctggaaaacctgagcctgaatccttatgagggcttgttgttacagttcccttttgtggcctgatccctctacacagaacctgccacccttctttcctctctcaggcctttggaattctggtctttctctccttattttgtttttgtttttaaacttttgcagattacatatgaattcttacactgggtgtttttgtcttcaaaataaaaaaaatcccccctgcaaaaaaaaaaaaaaaaaaaaaaaaaaaaaaaaaaaaaaaaaaa

SEQ ID NO: 4 - DNA, 8377 bp, Rattus norvegicus
aagcttgcttctaatttccaaaatgttagagttggtttttctggagaatctcaggattaacaaaacaaagacacaaaaagtgaagtccttgttgagatcaaaagtcactttagagtaggagagatggccgggtggttcacagtacttgctgcccttacagaggacctgaattcagttccctacatgggcagctcactaccccctgtgactcccagtcatgggggacccaatgccctcttctttcttctcaggcatctatatgcacgtggcatgaatacatacatacatttacttgggtctacagacacacataaaataaataatatgttttcgttgttgttgtcttttccagacagggtttctctgtgtagccttggctgtcctggaactccctctgtagaccaagctggcctcaaactcacagagatgtgccagcctctgcctcctgggtgctaggctcaaaggcctgcctgtaccatcactacctggaaaacagtaaatactttttaaagaggtattttaagaagtaaatacttttaacaataacaaaaaaggacaactagaactacggaggctttgaagaaccacccagcatgtttgtgtattctctgtcctgtaccaaaggtccagctcgccaggaattcggagggtaattgtccaagtcctcccctctaagcttcggtgggttatctgccgttatagatgtgtcagagccatgtgtcctgacttcagtctccggtactcaggcaccaggaagtcacagtgtggcccaggttctaggagagcaccaaggaggcgccagaggtgggtgctgcactccatgagaccctgggcagaaagcttcatcctccagctcacattccccgcctgtaagactggagccatctatcccccactgtgacacatggggatgaaccttggaaaagtcagtctaggcaggtgttagtaaaacaaacaggactatgtacataaaggattgagaaaaccagtcatccttagagttccgggaaggtagactcaagtatgtagacttggggcccggatgggtcaatccctgggtttgcccaccagcatctcacagctagaattgtctcctagttctggaaccttccagattcctgagaggctggctactcctgccttcctatcactgaatagagtagagggttctggaaaggacccaaaaaattctgctaagaggcaattcactcagatttcctttttttttttttaagagcttgccagagtcatatgatttatccccatacaggccaggccgaccagcctcggggcggggtgttgcaacaacagccagaacgccggctcagctgggcggccaccacgtgactcgagtacttaaagggctggcgcgggcagctgctcgctcacagtcgccagtcgcctccagagtttccgcctccaaccagcgagtggagcctgccggagcagagccatctcgagcggagcccggagcctgaagcgagcggagccagcctgaactagcccagtcccgcgatggagcgcccacagctcgacaggcaagcgactacttggggacgggaactgtgatgtcccgcgtcccttgtccagcctagaaatgcaggctgcgggaccaaggaacgagttgccgcccagtctacctgtagccatgggcagctggagggagaaaatgtgattcacccctctgacacgctggaggtgaccggatgggtgtgtcttgtagtatccaggtttgagggatgaacagtgctgagtcctggatcctggactcaccgttctagggggcgggggttggggagcttggccctcttggtggcgcggagtggtcacatcaattcccaacttcagacgacttgtaagacgcagggaggcagccgtagcagaactagagggtgttgaggctgggtgttttttgtttggggatgggggggacaggaagttttttgtttgcttggtgttttgttctgttttgctttggggtccctgtgtagatcaagttggccttgaactccagttcgcttccctttgcttcctgagtactgagattgaaggtgccccgccaccacacttggcttctattcctcctcctcctcctcctcctccttctcctcttcctcctcttcctcttcctcttcttcctccttctcctcttcctccttctcctctttgattttttttgagacggagcgcctctttgtagccctggatgtcttcactctgtagatcaggctggcctagaactcggagatctgcctgcctctgcctccctagtgctgagattacaggcctgcaccactctgtccagctcttttttctttttttccttttataaagccagttttatttttatatttttatttaaagcgtctcgagttacatttaatttatcgtgcatgtagagtggccacatatatgacacactaaatatgtggaggtcagagttcgacttgaagaaagtcacttctcagtctaccatgtggcttccaagaaagattggcttatcaggtggcaagtgcgtttcctggctgagcttacactgtctagtcctcacctccaacttttagcagtaaggattgaacttaggacttcaaacattctaggcaagcgctttacccataaggtacagtatcagcaccttttttatttaaagacagtgcctaactaagttgaccaggctggtctggaactcttgaatcttctgctgcagaacctctctaaataaagctgggttacatagactatttaatgcagaaactttgagtatagtgtatagttggttttggatgggttttttgtttgttttttgttttgttttgctgagactcttcctacatagccctgtgctgaaattcattcactatatagatagacccaggttggctttaaactaagagatctgcctacctccacctcctgcatgctggaattaaaggtgtgcaccatcacgtcctgctgcttgtaaaatctgtaactgaaattttttaagaaatatcttcaggttggatctaaaagctcaccaaagtatctacccttagccgggctgtggtggcccatgtctttaatcccagcactcaggaggcagagggaagcacatctctagagttctgggccagcctgctctacagagtgagatccaggacagccagggctacacagagaaacgctgtcttggaaagagaaaatctgcccttgagtatggtccttggactggcgtggctcttccctgaggtcaggacataccttgcctcctcttcactgggaataactggcgtaggaatcctccctggggttttggggactacgtcacatagtgtttaatgactgacatctgtttgcatggttaagatcatggcttggcctacattggaggctggagctattctgcaggtctaatcattcataggcatccccggctgactctcctctcttcctccacagcatgtcccaggatttgtccgaggccttgaaggaggccaccaaggaggtgcacatccgtgcagagaattctgagttcatgaggaactttcagaagggtcaggtgtccagggaaggctttaaggtacgtgacgtgtaggttgaccagaccactggggtatagcaggtatacccagggcttaagccaatagtttaatggggaggttggtgccaggagtgatgagggcctgtccaaggacgccatcttggtaatgctgaccctgtagctcagagaggaaagggtctgctggtggaggagctggtgcagtggggtggttaagtgaacacgtattctatgttggtcattttatgttcttgaggagtgcctaaggagaaggaacagagaggttaaagaggccatcctgtcagctttcagtggaaaagaccaaaaccaaagatggccaaatttcagagcttgcgctcttgacctggttaccgcagggcctcatacatgccggctaggttctctaccactgacccacaatccaagctcttaggttactagatcgggtctcactacacagttcaggaactctcaatactcctgcgtcagcctcccgggtatttggaccatgggagacccaaatctcctgacccaatttcaacttgcctctgaaggaaaggagaagtttgccttagccctgcagtgtcctgttcccacagtgagattggctacctcatttaacctcccaagccctcttgcctgaggaatggggttttgacagctgggctgaaatgcacagtagacacaatagaccctgtttcctgccagctgtggaactgatgtctgcagcaaacatcagacacagggaagagagcactgactgggtggggagtggagggtggtggagacaggagaccgtccttccagaagggttcttctagcgacaagttgattctgtttttttcagctggtgatggcctccttgtaccatatctatacggccctggaagaggagatagagcgaaacaagcagaacccagtctatgccccgctctacttccctgaggagctgcaccgaagggctgccctagagcaggacatggccttctggtatgggccccactggcaggaggccatcccttacacaccagccacacagcactacgtaaagcgtctccacgaggtgggaggtactcatcctgagctgctggtggcccacgcatatacccgctacctgggtgacctctcagggggtcaggtcctgaagaagattgcgcagaaggccatggccttgccaagctctggggaaggcctggcttttttcaccttcccgagcatcgacaaccccaccaagttcaaacagctctatcgtgctcgcatgaacactctggagatgacccccgaggtcaagcacagggtgacagaagaggctaagaccgccttcctgctcaacattgaggtgagccccactgttccttcaagccccattcattccagttattggaggaagggtccccagagtcccagttagtacaaggacttggggccagccagggtcctgttttgctgcttaactagctgggtgattgtgggcaaatctcttcatcctcaggttctaaatctccaaaatagggataataatagcatccatcttcaaggcgagaggggcaagcaagttgtggttcagtccctgtgggaggagtgatacaggggaagttccctgatgtcattgctctcttgtgtggtgggctgtgatcctctgacaatggtgtagctggacctggggtgcctgagtactggtgcatggccaggccagcacagtccaggcttcggatgctgacatgtcccaggtggcatttgcttccttttctttgatttgcattgatgtgttgcccacgggtatatctgtttggtgtcagattcgttgggggtggagctacagacagctgggagctgccacaggggaaccgggaattgaacccgcatcctctggagagcaactaacgctcttaatctttgaactatctttagctcggtgttttgagcaaggccccgaaaggcagagctgggggagtagactgtcctatttatactatgggtgctatggtggtcagggttccaatttgggacctcctgtctctccccactttcagcctgagagcctttaaaagttccaccaggcaagcacctgttagagaagtaaccttcagatgtcctaaattcatctaggcccgttcacctgaggccttccgaggacatgatataaagaatgagtttagaagtggggaagtgatggcccattcctttaatctcagcactcagaaggcagagacaggaggatctctgtgagttccaggtcagcttggtctacacagctagttctaggacagccgaggctacacaaagaaaccctgtcttgaaaaacaaaacaaaacaccaacaagaaaagaggattagacacaggcccagctgagaactgggtctccatcctgacccctagtcgatctcaagaaagccctgctccaggagctgtggttccctgacctgcaaaagcagcgaccatactccattacacgacacaccgcctagcaaggaggagctcaagtacagtcatgaagagagcacgggatgtaggctgcacggctcccacacattcgtgtgttatttccgaagaggcaaactggttctcctcatagggatacagaaaagctcctgaagtctttattcagggcagccgttactggtggggtccaagcgagggtacagacagtatcggggacgcacaggcagggagccaactaaacctgtcacatctcatgagataagtgaaagacgcggtatgcgatagtttcctcaagtcgtgtcatttgtacaatatttcccaacaggcttcaagctgtgccttcccctgggtccccccacattgtgagtatcactaacaggcagtgtgtcttgtcttgtcttcagctgtttgaggagctgcaggcactgctgacagaggaacacaaagaccagagtccctcacagacagagtttcttcgccagaggcctgctagcctggttcaaggtaaagatggcctcttggcttctgctctccttagcccagctggaatttctttactcttctccattctgaacgttttaaattagagatagggtgcagacaaaagagcccagggtaagtctgtagtatgactgtgggtaagctatcagacttttctaccttagtttctccacattagaaaatcaggaggttgggggctgtgtagctcgttggctgaggggcatataaagccctcagaagtcactggcacagagatcctctcatttagatcccgcctcccacttcttttcccccaaggcagagccatgctgcatgacccaggctggtctcaaaccctgggagtcaaacaatcctcctgcttcaatctttcataaaaatctagggtgtgtttaccttttgaggcccactcaaggctcccatgcaggccccatatgaggcaaggatttgggaagctatttctagcgctgaccctccaggagacacgacggagttcatgtaattactggagtgtcagcccttttcctgtgccttgatacactacccttggagcaggagtaagggttcttaaaaagaccaaagtggcagaaaatgtgccttccctgagcctatcacccctgggacagtatggcccagggagtgggtgactgaaactgagcagggcaggttacgtgaagcttgattgcttgaatgggcccaccttttgcccaaggctctcccaagggaggcaggacccgccaccttgtcattagtggtactgtttctggtgggctgaaatatggtcagtgctgatagaattagttacctgtttgggtctggagtctttgacctgctgcggcctggcggagcacaggaggaacagcgtgggggagttggtgagctcctatctttgaaggtgtttaagcagtggctagtggttcaagctgtagcagaatcctggccttggactggaactgtagcagacacggcaccgagtgtcctctgctctgtgcagtgtcatccaccccaggctgtgtgtttgaccttgtctgtgtttctcctacagatactacctctgcagagacgccccgaggaaaatcccagatcagcactagttcatcccagacaccgctcctgcgatgggtcctcacactcagtttcctgttggcgaccgtggcagtgggaatttatgccatgtaaatgcagtgttggcccccagaggctgtgaactctgtctcatgtagccttctctctgcaggggagaatcttgcctggctctcttttcttgggcctctaagaaagcttttggggttcctcgcccccttcctgtgtcttcctttgtctctctggaatggaaggagatgcctggcacatttccctcaccaaaagcacagccaggggcctgaacttggaaaccagcagccccaaatcctgcaacagagccccagaactgtggtcggtagaggcggctgttctgagcccagtgcccttggtagtcggcgtccatgttgactgaccatgactgctttcccccagcgtcatggccactttgatatcagtgtgcagagattttctgtcttgtgtttctgtcttgtttttgttatttccccagttctaccagtgtaatggtattttttgttgtatcgattgtttttttattctaaccaagtagggctgtcttttgaggggtgggtggtatttaataattgtaaccttggtctctaacttctgtgtgaaataataaatggcattatctaataatcaccggaagttcgttcatgtctctgccgtccacgaacagggccctgctcctcctgtctcttcagtgtctggcaggaggagggcaggtggtggtggcatcagtagctttgacggtccactgtgtctgagagttggaagtgagcttcaagcatttgggc

SEQ ID NO: 5 - DNA, 386 bp, Rattus norvegicus
tttttttttttttttttaagatatgacgactttattctgtaaacatatccaagggcccaaccccaggccaaaagctctgttaccccttgtggctgtctttatgaactgccaagcccacccttatcaccaacacaaggaactcttcgaagttaattgcgttgtcactattgacgtccaattctttgaacaagctttcggtatttttattctgcacaaactgagggcactcagtagtgaccattttcctgaagtcatccctgtaaagggcatggtgattcccttttataccaaaataattgtggtaaacttcaatgacgttgctcaaggccttctccaattcaattgccattgtcgataaaaatttcctttcacacaaggtctggacc

SEQ ID NO: 6 - DNA, 3471 bp, Rattus norvegicus
ggcacgaggaccggctgaggattttatggttcttaagcggacttaagagcgttgtttcggattgttaagattcccgtttgctgggttttcctccctcaatcgtgctctcccgcggctgcctggggactggctcggcgaaggaggatggagagggggctgccgttgctgtgcgccacgctcgcccttgccctcgccctggcgggcgctttccgcagcgataaatgtggcgggactataaaaattgaaaacccggggtaccttacatctcccggctaccctcattcttaccatccaagtgagaaatgtgaatggctaatccaagctccggagccctaccagagaatcatgatcaacttcaacccacatttcgatttggaggacagagactgcaagtatgactatgtggaagtgatcgatggagagaatgaaggtggccgcctgtgggggaagttctgtgggaagatcgcaccttcacctgtggtgtcttcagggccatttctcttcatcaaatttgtctctgactatgagacccacggggcaggattttccatccgctatgaaatcttcaagagagggcccgaatgttctcagaactatacagcacctactggagtgataaagtcccctgggttccctgaaaaataccccaacagcttggagtgcacctacatcatctttgcaccaaagatgtctgagataatcctagagtttgaaagttttgacctggagcaagactcaaatcctcccggaggagtgttctgtcgctatgaccggctggagatctgggatggattccctgaagttggccctcacattgggcgttactgtgggcagaaaactcctggccggatccgctcctcttcaggcattctatccatggtcttctacactgacagcgcaatagcaaaggaaggtttctcagccaactacagcgtgctgcagagcagcatctctgaagatttcaagtgtatggaggctctgggcatggaatctggagagatccattctgaccagatcactgcatcttcccagtatggtaccaactggtctgttgagcgctcccgcctgaactaccctgaaaacgggtggacaccaggagaggactcctacagggagtggatccaggtggacttgggcctcctgcgattcgttactgctgtggggacacagggtgccatttccaaggaaaccaagaagaaatattatgtcaagacttacagagtagacatcagctccaacggagaggactggatcaccctgaaggagggaaataaagccattatctttcagggaaacaccaatcccacggatgttgtctttggagttttccccaaaccactgataactcgatttgtccgaatcaaacctgcatcctgggaaactggaatatctatgagatttgaagtttatggctgcaagataacagattacccttgctctggaatgttgggcatggtgtctggacttatttcagactcccagattacagcatccaaccaaggagacaggaactggatgccagaaaacatccgcctggtgaccagtcgaaccggctgggccctgccaccctcaccccacccatacatcaatgaatggctccaagtggacctgggagatgagaagatagtaagaggtgtcatcattcaaggtgggaagcaccgagaaaacaaagtgttcatgaggaagttcaagatcgcctacagtaacaatggttctgactggaaaatgatcatggatgacagcaagcgcaaggctaagtcttttgaaggcaacaacaactatgacacacctgagctccgggcctttacacctctctccacaagattcatcaggatctaccccgagagagccacacatagtgggctcggactgaggatggagctactgggctgtgaagtagaagtgcctacagctggacccacgacacccaatgggaaccccgtggacgagtgtgacgatgaccaggccaactgccacagtggcacaggtgatgacttccagctcacaggaggcaccactgtcctggccacagagaagccaaccattatagacagcaccatccaatcagagttcccgacatacggttttaactgcgagtttggctggggctctcacaagacattctgccactgggaacatgacagccacgcgcagctcaggtggagggtgctgaccagcaagacggggcccattcaggaccacacaggagatggcaacttcatctattcccaagctgatgaaaatcagaaaggcaaagtagcccgcctggtgagccctgtggtctattcccagagttctgcccactgcatgaccttctggtatcacatgtccggctctcatgtgggtacactgagggtcaaactgcactaccagaagccagaggaatatgatcaactggtctggatggtggtcgggcaccaaggagaccactggaaggaagggcgtgtcttgctgcacaaatctctgaaactgtatcaggttatttttgaaggtgaaatcggaaaaggaaacctcggtgggattgctgtggatgatatcagtattaacaaccacattcctcaggaggactgtgcaaaaccaacagacctagataaaaagaacacagaaattaaaatagatgaaacagggagcaccccaggatatgaagaagggaaaggcgacaagaacatctccaggaagccaggcaatgtgcttaagaccctggaccccatcctgatcaccatcatagccatgagtgccctgggggtgctcctgggtgcagtctgtggagttgtgctgtactgtgcctgttggcacaatgggatgtcggaaaggaacctatctgccctggagaactataactttgaacttgtggatggtgtaaagttgaaaaaagataaactgaacccacagagtaattactcagaggcgtgaaggcacggagctggagggaacaagggaggagcgcggcaggagaacagtggaggcgcagggactctgttactctgctttcactgtaagctgggaagggcggggactctgttactccgctttcactgtaagctcggaagggcatccgcgatgccatgccaggcttttctcaggagcttcaatgagcatcacctacagacacaagcaggtgactgcggtaacaacaggaatcatgtacagcctgctttcttctcttggtttcgtttgggtaatcagaagccagttgagaccaagtgtgactgacttcatggttcatcctacttggccccctttttcctctctttctccttaccctgtggtggattcttctcggaaactgcaaaatccaagatgctggcactaggcgttgttcagtgggctctttcgatggacatgtgacctatagcccagtgcctagagcatattagcataaccacatttcaggggacaccaatgtccgcttttgcatcgctacgtgcagcgagcacaggaaaaagaaaaaaaaaaaaaaaaaaactcgag

SEQ ID NO: 7 - DNA, 3987 bp, Rattus norvegicus
agtatggagagaaggtcgtttaaaaaggcagatgtccctttaaggtttgctttgctgctgcccgtggactttagcctaaacagggtcccgcgaagttggctttatttgtccatgtctcggacacagcctgggtagctgccagtgagatttcagggacggagcgcgcaaaggggggggaaatgtggcaatccatctgggatgtgagacgcgtggagagggcttagcagcatttgaccaaaacacaggaaatcactcctccacagctcctgggcgcagcagcggctggggccactgccggacaccctcggagaccacacgagtgacccagagcgcaagtcgccagcgtcccggttctgcctgttcctgccagctcctgcccacgaaccggcacgtagctggttccagcagccgctccagcaatggtccccaggcgtcctgccagcctagaggtcactgtagcctgcatatggcttctcacggtcatcctaggcttctgcgtctccttcaatgttgatgtgaaaaactcaatgagtttcagtggcccagtagaggacatgtttggatacactgttcaacaatatgaaaacgaagaaggcaaatgggttcttattggttctcctttagttggccaacccaaagcaagaactggagatgtctataagtgtccggttgggagagagagagcaatgccttgcgtgaagttggacttgccagttaacacatcgatccccaatgtcacagaaataaaggaaaacatgacatttggatcaactttagtcaccaacccgaatggaggatttctggcatgtgggcccttgtatgcctatagatgtggacatttgcattatacaactggaatatgttctgatgtcagtcctacatttcaagttgtgaactcctttgcccctgtacaagaatgcagcacccagctggacatagtcatcgtcctggatggctccaacagcatctacccctgggaaagtgtcatcgcctttttaaacgaccttcttaagaggatggatattggccctaagcagacacaggtcgggattgtacagtatggagagaatgtaacccatgagttcaacctcaataagtattcatccacagaagaggtccttgtcgcagcaaacaaaataggccgacagggaggcctccaaacgatgacagcccttggaatagacacagccaggaaagaggcattcactgaagctcggggtgccaggaggggagttaaaaaagtcatggttattgtgaccgacggagaatcgcatgacaactatcgcctgaaacaggtcatccaagactgcgaggacgaaaacattcagcgattttccatagctatccttggccactataacagggggaacttaagcactgaaaaatttgtggaggaaataaaatcgatcgcaagcgagcccacggaaaagcacttcttcaatgtctcggatgagttggccctggtcactattgttaaagctctgggagaaaggatattcgctttggaagcgacagctgaccagtcagcagcttcatttgagatggaaatgtctcagactggcttcagtgctcactactcccaggactgggtcatgcttggagcggtgggagcctatgactggaacggaactgtggtcatgcagaaggctaaccagatggtcatccctcataacaccacctttcaaactgagcccgccaagatgaacgagcctctggcttcttatttaggttacacagtgaactcggccaccatccctggagatgtgctctacatcgctgggcagcctcggtacaatcatacgggccaggtcgtcatctacaagatggaggatgggaacatcaacattctgcagacactcggcggagagcagattggttcctactttggtagtgtcttaacaacaattgacatcgacaaagattcttatactgatctgcttctcgtcggggcccccatgtacatggggacagagaaagaggaacagggcaaggtgtacgtgtacgctgtgaatcagacaaggtttgaatatcaaatgagcctggaaccaattaggcagacctgctgctcatccctgaaggataattcatgcacgaaagaaaacaagaatgagccctgcggggcccgcttcggaacagcaattgctgctgtaaaagacctcaacgtggatggatttaatgacgtcgtgattggagctccgctggaagatgaccacgcaggagctgtgtacatttatcatggcagtggcaagaccataagggaggcgtatgcacaacgcattccatcaggtggggatggcaagaccctgaaatttttcggccagtctatccacggagagatggatttaaatggtgacggtctgactgacgtgaccattggaggccttggtggagcagccctcttctgggccagagatgtggctgtagttaaagtgaccatgaattttgaacccaataaagtgaatattcaaaagaaaaactgccgtgtggagggcaaagaaacagtgtgcataaatgctacaatgtgttttcatgtgaaattaaagtctaaagaggactcaatttacgaggctgatctgcagtaccgtgtcacccttgattcactgaggcagatatcacggagctttttttctggaactcaggaaaggaagattcaaagaaatatcaccgttcgagaatcagaatgcatcaggcactccttctacatgttggacaaacatgactttcaggactctgtgagagtgactctggattttaatctcactgatccagaaaatggtcctgtacttgatgacgctctgccaaactcagtccacgaacacattccctttgccaaagactgtggaaacaaggaaagatgcatttcagacctcactctgaatgtgtccaccacagaaaagagcctgctgatcgtcaagtcccagcatgacaagttcaacgttagcctcaccgtcaaaaacaaaggagacagtgcgtacaacaccaggacagtggtgcagcattcaccaaatctgattttttcgggaattgaggagatccaaaaagatagctgtgaatctaatcaaaatatcacttgcagagttggatatcctttcctaagagcaggagaaacggttaccttcaaaataatattccagtttaacacatcccatctctcggaaaatgcaatcattcacttaagtgcaacaagtgacagtgaggagcccctggaatctcttaatgataatgaagtaaatatttccatcccagtaaaatatgaagttggactgcagttttacagttctgcgagtgaacatcacatttcagtcgctgccaatgagacgatccctgagtttattaactccactgaggacattgggaatgaaattaatgtcttctatacgattagaaagagggggcatttcccaatgccagaacttcagctgtcaatttcattccccaatttgacggcagatggttatcctgtactgtacccaattggatggtcatcttcagataatgtgaactgtagaccccggagccttgaggacccctttggcatcaactctgggaagaaaatgacaatatcgaagtctgaggttctcaaaagaggcacaatccaggactgcagtagtacgtgtggagttgccaccatcacgtgtagcctccttccttccgacctgagtcaagtgaatgtctcgctcctcctgtggaaaccgactttcataagagcacatttttccagcttaaaccttactctaagaggagaacttaagagtgaaaattcatcgctgactttaagtagcagcaaccggaagcgagagctggctattcagatatccaaagacgggctcccaggcagagtgccgctgtgggttatcctcctgagcgccttcgcggggctactgctgctaatgctccttatattggctctgtggaagattggattcttcaaaaggccactgaagaagaaaatggagaaatgaaaggtttcatagaaaaaaaaaaaaa

SEQ ID NO: 8 - DNA, 426 bp, Rattus norvegicus
ttctctcaagacttggtagttcaggggaagatccatccccagtggctcttcgcttctttctaatactggggccatcgttttgctcagtaccagaggtacaactgctgtcagtggctgcatcagctaagctggagactgcatgacctgaagatgatgcagttgatgaaagtgggtttccttctccagtgctctcctgggtgtggtgctctgatagtggggtagtcctctcctggctatgcaggtctctggctgaatgcatgaaaggcattatgtgatcagatggtaactgtgcgctatgtcgccgctgggtaggaccggtgccaaccctttcacctgttctgacagagggagacagaggtgccacttccggttcaagatacatggctgccttctgagatgcagtactaggtgaaggactctgctggg

SEQ ID NO: 9 - DNA, 540 bp, Rattus norvegicus
tttttttttttttttttcttcttcttaataagggataaaagatcacctcaccctcttccggttacacagaagctttgttttcaaaattccatctgtggtctccatctgcccccattagatgagttttttttttgtcacggcccatagctaattccctccatcagataacagtgggaaacgtctacaatgaaccatgtttggaagaagacgcccaaattcaccaaacatgagacttaggagccaatcacctgcgtccaactggcaaaagacatatcccttaggtagcagctactgtagtcagcaggagtaggcaacgtagagaccccaccacgctcagtcttgggaacacgtggtgaggcatcaggacgcttcttacatccaaaatggttcagtcgtgtggttgggtggttctgcataacctccccagcgtgtcccttctcatttctgattccttcttgggttcaatgctttgcaaactactcatgccgcaccggcatccatggtttcttgcaattctactaaagggggcatatgcactag

SEQ ID NO: 10 - DNA, 647 bp, Rattus sp.
ggattcttttcttttattattggcatcagctggactatatgtggccttaaacatcatgcactggacaggaaagaaagaacgagaaaaaaggacacaggaagggaacacgagtagcggcgagattccgtaataaaaactgtaattcattcaataggttaagttttggcattatgaaatcaaacaccccttcccacccccaaaagttgaaacttaagtgagagggtcgcccgtgggacccagccaggtgttctgcgttccctcacaggctgtgacaggcagcgtgtgggtgtaattcttgtgaacggtctcaacttttcactttctttaaataatctcttttgcttcttagacgttccggttcactggggtgacataattgcggggaaacatgccggtctgcccatggcaagcccctttccaccaattgggatctgagttatccatgacatgaatgaagtctcctcggcgaaagcccagctcaccatcctcctgggggtcaaagtcaaagagtgcctggacgtacgttggctgtggcacctgttctatgtcccggaggaatatctgctggttcctggacacggatgttgatctgtggtaatctaccagctcattcaaagaattaaacttcaccacccacaggaagtact

SEQ ID NO: 11 - DNA, 531 bp, Rattus sp.
cgcaacgggtttgccgtcagaacgcaggtgttgtgaaagccaccgctaattcaaagcaaaaatgggaaaggaaaagactcacatcaacatcgtcgtaattggacacgtagattccggcaagtccaccacaaccggccacctgatctacaaatgtggtggaatcgacaaaagaaccattgaaaagtttgagaaggaggctgcggagatgggaaaaggctccttcaagtatgcctgggtcctggacaaactgaaagctgagcgtgagcgtggtatcactattgacatctccctgtggaagtttgagaccagcaagtactatgtgactatcattgatgccccaggacacagagacttcatcaagaacatgattacgggcacatctcaggctgactgcgctgtcctgattgttgctgctggtgttggggaatttgaagctggtatctccaagaacgggcagacccgtgagcatgctcttctggctacactttgggtgtgaaacaacttaattgtggtgtcaacaaaatggattcc

SEQ ID NO: 12 - DNA, 528 bp, Rattus sp.
caaccacagaagagtttattactccaatgattcgcacagctgggttgcaggtctaagggttgcctgccttcctggaggcactcccccacttccccctctgggggaattacacttggccacatacatctctgcaaactgcttcagtgtgaaggcccacccatgcaggccagagccaaagcctacagtgcccaggacaggatcgatcatgatattgcccatgggtccactctcgccctcgccataggtggagatgatgacattgacgttctccacgatgcgctggaaggtctggtagagctcctcaggctccagttgcagttccagcagggcccggtccatcttgttcatcatcagcacgggcttgatgcgctcagcgatggcctgccgcagcactgtctctgtctgcacacagacaccagacacacagtccaccaccaccagtgctccatcagtgacacgcagagcagcggtcacctctgaggagaagtccacatgacctggagagtcaatgaggttgatcctcgtgcc

SEQ ID NO: 13 - DNA, 1136 bp, Rattus norvegicus
gggcttcttggacgtttttgggaggggacagcaagggaaggtccttctgcctctagggacccagacttccgctttctgcagacagcagcaggctctgggctctgggaatccactgctgtctggcctagaagcatcatagaacacgaggattccatacacaggaggcccctgaagctgagctgagctgatgaagacacagtggagtgagatcttgacacccctgttgctgctgctcctgggtttgctccatgtctcctgggcccaaagcagctgtactgggtcccctggcatccctggggtccctggcatccctggggtccctggctctgatggcaaaccaggcactccagggataaaaggagagaaagggctccccggactggctggagaccatggtgagttaggagagaaaggggatgcagggatccctgggatcccaggcaaagttggccccaagggtcccgtcggccctaagggtgctccaggcccccctggaccccgcggtcccaaaggtggctctggagactacaaggctacccagaaagtagccttctctgccctgaggacggtcaacagcgccctgcgaccaaaccaggccattcgcttcgaaaaggtgatcaccaatgttaatgataactacgagccgcgcagtggcaagttcacctgcaaggtacctggcctctactacttcacctaccacgccagttcccgcgggaatctgtgtgtgaacatcgtgcgcggccgcgaccgagaccgcatgcagaaagttctcaccttctgcgactatgcccaaaacaccttccaggtcaccacgggtggggtagtcttgaagctggagcaggaagaggttgttcacctgcaggccacagacaagaactccctgctgggcgtcgagggagccaatagcatcttcactggctttctgcttttccctgacatggatgtatgatcacggggtcaaatcactcctatccaaaacctcctccctgccagtaatcctccctggaccccagacactgccctttgactgcccaaagccctgaccagagccctgtagatgttacagaatgggtaaataaactcttcaaggccaagaaaaaaaaaaaaaaaaaaaaaaaaaaaaaaaaaaaaaaccc

SEQ ID NO: 14 - DNA, 3256 bp, Rattus norvegicus
cacgaggcgactgggggcgcggaagggaggcgaggttccgggggtggggcggcccggaggcgagcgggcgagcggggccgccgagcggcggcgagagggcaggcggcggcggccgaggcggagagagcggcgcggggtgcggccgccgagaggagctcctcccccgtcccctcaccccctcagctgagctcgagtcggggcggggcggggtacgtggagcggggccgggcggggagctgctccgagtctggccggagcgcacccggggcgcccgcgttcgccgctcgcgggaactttgcggcggacggtgtggaggccgcggaggggtgcatgagcggcgcagggcggcgcacagagcggcggctgtccggtccggcccctgctggagtcgccgccggaggaggcgagcgaggagccaggtgtgtgtgccttcttcctgcttctggaaaaaatgccggtccggaagcaagatacccagagagcattgcatctgttggaagaatatcggtcgaaactaagccaaaccgaagacagacagctcaggagttccatagagcgggttattagcatatttcagagcaacctctttcaggctttaatagacattcaagaattttatgaagtgaccttacttgataatccaaaatgtgtggatcattcaaagcagtgtgaaccggtccaacccgggaatccttgggagagtggcagcctttcaagtgctgctgtgacttcagagagcctgcccggcggccttagccctccagtagagaaataccggtatcaggacgaagaggtgctgccttcagagcggatttccccgcaagtcccaaatgaggtgctgggtccggagctggttcacgtctcggagaagagcctgtcagagattgagaatgtccacgggtttgtctctcactctcatatctcacccataaagcccacagaagctgttcctccctcctctcccattgtccctgtgacccctgccttgccagtccctgctgagagtcctgtcgtcctgccctccacaccacaggcaaatcctcctcctgtcctggtcaacacagacagcttggagacaccaacttatgttaatggtactgatgcagattatgaatatgaggaaatcacacttgaaaggggaaattcaggcttgggtttcagcattgcaggaggtacagacaacccacacattggagatgactcaagtattttcatcaccaaaattatcacgggaggagcagctgcccaggatggaagattgcgggtaaatgactgtatactgagagtaaatgaagcagatgttcgtgatgtaacccacagcaaagcagttgaagcattgaaagaagctggatcgatagtgcgattatatgtgaaaaggcggaaggccttcagaaaaaatcatgaaataaaacttattaaaggccctaaaggtcttgggttcagcattgctggaggtgttggaaaccagcacattcctggggataacagcatctatgtaaccaaaataattgaaggaggtgcagcacacaaggacggcaaacttcagattggagacaagcttctagcagtgaacagtgtctgtttagaagaagttactcatgaagaagcagtgactgccttaaagaatacatctgattttgtttatttgaaagcggcaaaaccaacaagtatgtatataaatgatggctatgcaccacccgacatcactaattcttcttctcagtctgttgacaaccatgttagcccgtcttcatacttgggccagactccagcgtcaccagccagatactcacccatttctaaagcagtgcttggagatgatgagatcactagggaacctagaaaagttgttcttcatcgtggctcaacgggacttggtttcaacattgtgggaggcgaagatggagaagggatttttatctctttcatccttgctgggggacctgctgacctaagcggagagctcagaaaaggagatcgcattatatcggtaaacagtgttgacctcagagctgcaagtcatgaacaagcagctgccgcattaaaaaacgcaggccaagctgtcactattgtcgcacagtatcggcctgaagagtacagtcgttttgaagctaaaattcacgacttgcgggagacgatgatgaacagcagtgtcagctcagggtcagggtctctccgaaccagccaaaagcggtccctctatgtcagagccctttttgattacgacaagactaaagacagtggccttcccagtcaagggttgaacttcaaattcggagatattctccatgttatcaatgcttctgatgacgagtggtggcaggccagacaggtgaccccagatggtgagagtgacgaagttggagtaattcctagtaaacgcagagttgagaagaaagaacgagcccgattaaaaacagtcaaattcaattctaaaacaagaggagataaaggggagatccctgacgacatgggatcaaaaggcctgaagcacgtaacttctaatgccagcgatagcgaaagtagttaccatgaatatggctgctcaaaaggtggtcaagaagaatatgttttatcgtatgagccggtgaatcagcaagaagttaattatacccgaccagtcatcatattaggacctatgaaagacagagtaaatgatgacttaatctcagaatttcctgacaaatttggatcctgtgtccctcatacaactagaccaaagcgtgactatgaggtggatggacgagattaccattttgtgacttcaagagaacagatggagaaagatatccaggagcataaattcattgaagctggccagtataacaaccatctatacggaacgagtgtccaatctgtgcgagcagtagcagagaagggcaagcattgtatccttgatgtgtctggaaatgccatcaagaggttgcagattgcacagctctatccaatatctatttttattaaacccaaatccatggaaaatatcatggaaatgaataaacgcctaacagacgaacaggccagaaaaacgtttgagagagcagtgagactggagcaggagttcactgagcatttcacagctattgtccagggagacacgctggaggacatttacaatcaagtgaagcagatcatagaagagcagtctggtccttacatctgggtcccagcaaaagaaaaattatgaagacagatgttgctctttctttttccatggtctcatctctctgccctgctctggaggtctgtcttcagc

SEQ ID NO: 15 - DNA, 1699 bp, Rattus norvegicus
gaattcggcacgaggtattttttctctcttcacttctccttttacacaaatagacatagacttctgggttaccagactgtgctcgccactttaaagataataagcaaattctacctgaagtactgagggacactgccttctaaagggcatactatcccagcatgggcatgagcagcctgaaattgctgaaatatgttctgtttttctttaactttcttttttgggtctgtggctgttgcattttgggctttgggatccaccttctggtccagaatacctatggaatactcttccgtaatctccccttcctgacacttggcaatgttctggtcattgtgggctccattatcatggtagttgccttcttgggctgcatgggatcaatcaaggagaataagtgcctgcttatgtcgttctttgttctgctgctgcttattctccttgctgaggtgaccttagccatcctgctctttgtgtatgaaaaaaagatcaacacgttagtggcagagggcctgaatgacagcatccaacattatcactctgacaacagcaccaggatggcatgggacttcatccagtcacaactgcagtgttgtggtgtaaatggctcaagtgattggatcagtggtccaccatcttcctgcccatcaggtgcagatgttcagggttgttataaaaaagggcaggcgtggtttcactccaatttcttgtatattggaatcgttaccatctgcgtatgtgtgatacaggtgctggggatgtcctttgctctgacactgaactgccagattgacaaaaccagccaggctttagggctgtgatttacacctaccttctgcttaagtgatttatttctctctagaaagtcaaagcacccattccataaggagttaaacaattacgtgcctcactggcattttgactttctcttattccttctttgactggacctctgtcttatacacatccagggaagagaacatttgtcatggacttcccacatcaagcagaagacaaacattaaccaactgatagcagtaaccatatcccttaaagatgatgaaacatacctgggtaattttttaaaagagacactgtagcactgtaatttgatatccactgctagctgcaggtgtgatatatctcagaatactggcctaccacatgtaatccattgagtttagccccaaatccaataaaaagaggtccaagtttaaatgttagactcttaacaattgtaaaatcaatctctaccttctaaatcttgctgctgtcactctacaaagtcacataggagacaagctgaaggaaaatttggagtcccattcattagataattgacatgtccagttcccttttgaacacagtccttgggaataggaatcatacagagatcttttatcactggaaaaacattctgatttctttgttaaaatgtattattgattttgttacatcatccacacatccatccatccatccatccatccatccatccatccatccataaaagactaattattctcatcctaccatcagccagtttgcagctttgaattacattgccaaagaacaactcagaagaattaatatctccagtgtgttagctgataatcatatccatgttgatgttgttgttatttctctattaaataaggttctgttaatgtaccattttaacctgtctcgtgccgaattc

SEQ ID NO: 16 - DNA, 578 bp, Rattus norvegicus
ctttgaagagatcagtgactctgtaaccttgcaactgcattttgatggttcctttaaactcgcggacttgtactctctcctcggatggctagctgattacttctgtgtagcagcttacaaatgattcgaagtgttgaaactttctgagtctcaatgaactatttctcacacaactcagatactgctgttcatgcatgaattagttgaaccaggccacatagaaagcattgtaattgtacctgtgagttggcaagaagcaagtgctcttccttgattctgctgcacgaggaggagaatgggctgcgatcggaactgtgggctcattactggagccgttattggtgctgtcctggctgtgtttggaggcattctcatgccggttggagacctactcattgagaagacaatcaaaagggaagttgtccttgaagaaggaaccattgctttcaaaaactgggtgaaaacgggcaccactgtgtacagacagttttggatctttgacgtgcaaaacccagaggaagtggcaaagaatagcagcaagatcaaggttaaacagagaggtccttacacatacagag

SEQ ID NO: 17 - DNA, 474 bp, Rattus norvegicus
attatgtgccgaaaggggaaggttggaccgcctggcctggacatcggaaggaaggaagatcagctgattgtccacatatttcaccctaaggtcaatgtgagtcaggaaaccatgtttggtgacggaaatacctgttacacattcgactacactgtgtttgtgaaacattacaggagtggggagatcctacatacagaacatagcgtcctaaaagaagattgtagcgaaactctgtgtgagttaaacatctcagtgtccacgctgaattccaattactgtgtttcagtagttggaaagtcgtctttctggcaagttaatacagaaacatcaaaagacgtctgtatcccctttctccatgatgacagagaagaatcaatttggatgctgctcgttgctcctcttctcttccttacaatagttgtcccggcacttgtgtgttgttacattaagaagaatccatttaagagaaaaagc

SEQ ID NO: 18 - DNA, 3709 bp, Rattus norvegicus
cgcgctcaccgccgatctcccatccttgctctgcagccgcggcccatggagcccgccggcccggcccctggccgcctagggccgctgctgttctgcctgctgctctccgcgtcctgtttctgtgcaggagccagcgggaaagaactgaaggtgactcaggctgacaaatcagtgtctgttgctgctggagattcggccactctgaactgcactgtgtcctccctgacgcctgtgggacccattaagtggttcaaaggagaagggcaaaatcggagcccgatctacagtttcataggaggagaacactttcctcgaattacaaatgtttcagatgctactaagagaaacaatatggactttagcatctgtatcagtaatgtcacccctgaagatgctggcacctactactgtgtgaagttccagaaaggaatagtagagcctgacacagaaattaaatctggagggggaacaacgctctatgtactcgccaaaccttcttcaccggaagtatcgggcccagactccaggggctctcctggacagacagtgaacttcacctgcaagtcttacggcttctctccccggaatatcaccctgaagtggctcaaagatgggaaagaactctcccatttggagaccaccatctccagtaaaagcaatgtctcctacaacatctccagcacagtcagcgtgaaactaagccccgaggacattcattctcgggtcatctgcgaggtagcccacgtcaccttggaaggacgcccgcttaatgggaccgctaacttttctaacatcatccgagtttcacccaccttgaagatcacccaacagcccctgacgcccgcgagccaggtgaacctcacctgccaggtgcagaagttctaccccaaggctctccagctgaactggctggagaatggaaacttatcacggacggacaagcccgagcatttcacagacaacagggatgggacctataattacacaagcctgttcctggtgaactcatctgctcacagagaggatgtggtattcacgtgccaggtggagcatgacagtcagccagcgatcaccgaaaaccataccgtgcgggcatttgcccactcgagtagtggaggcagcatggaaaccatccctgataataatgcttactacaactggaacgtcttcatcggtgtgggtgtggcgtgtgctttgctagtagtcctgctgatggctgccctctacctcctccgaatcaaacagaagaaagccaagggctcaacttcttccacacggttgcacgagcccgagaagaatgccagggaaataacccagatccaggacacaaatgacatcaacgacatcacatacgcagacctgaatctgcccaaagagaagaagcccgccccccgggtccccgagcccaacaaccacacagaatatgcaagcattgagacaggcaaactgcctaggccagaggataccctcacctatgctgacctggacatggtccacctcaaccgggcacagccaacccccaagcctgagccatccttctcagagtatgccagtgtccaagtccagaggaagtgaatggggctgtggttggctctaggccccatccccacaagttttcttgtcctacatggagtggccatgatgaggacaaccagccagccagccctgtctccagaaggccaggtggcacaggtcctaggaccaggggtaagggtggcttctgtcttccctccgtggctctccaacacctcttggacacccatgtccccttcttctggagctgggtgttgcagaaccagagggggaactggagaaagctgcctagaatccaagaagcgttgtgcctcagcccatcacactgggtctggatcctggtcttggcaacccccaggttgcttccttgatgctccagcgcctggtcttctgtgtggagaagagttcaccatctccatccaacttgagcttcggggccagactccctttagatcagaccgccccatgtgtggaagaactacaccaggagtcaacaagttttcacatgtgtgaagaactacaccaggagtcaacaagtttacgccaacagtgctagcctccccacctcccaggctgacgagccctgaggagaaggaaccctcttccccctagaccagcagagactccctgggcatgttcagtgtggccccacccttccagtcccagctcgcttcctccagctagcactaactcaacagcattgctctgtggacgcctgtaaattattgagaaatgtgaactgtgcagtcttgaagctaaggtgttagaaaatttgatttgtgctgtttagttgttgttgggtttcttttcttttcttttttttcttcctttttctttcttctttttttcttttcccccttaaaacaacagcagcagcatcttggctctttgtcatgtgttgaatggttgggtcttgtgaagtctgaggtctaacagtttattgtcccggaaggattttcttatagcagaaacagattttttttccaattcccagcaccctgaggaccaagaaggatccctctgttgtcattttcagcactcagcgtcactgggatgagccaggctctgtccccacagctggcccttggcctccatggctactgtggtaagtgcagccttgtctaatccagtgctgacgttggccattcctcattgaggagagaaggtcagtgacaaactcacaagcactgcagaggcatacggagagaagggacgctcggccagcacccggtattccagcgctctgaggtaatcagtgcaaggagtctgttattaccatcagacctcagcaggatcatactggaacagaacctgatcatacctgtgacaacacagctgtcagccagggcaaaccaccccactgtcccagagtctgggcagaggctctgacccccacccttcaaactggatgtcggggcctggctgggcccaatggcaagcagatgttgcaaccctagctatctggtcttaacatgcagctcagtaagttgaggcgctaatgtccccccatgccgggggattcctggttccggctcttcaagtaagaagctgattcaacctgcctgtttctgtaggtgtgacagggatgtcaggaaaacagccaggactcatctctatagggctggtgacctgatacttcccataaaggcatccaggagttagctgacccaatagtcagagttgacctcactggcctagcaaaccgtaacttgtctttggcccagccatggtcttgggctgtcttctaattccaaagggttggtaggtaaagatccatcctcttcccctctgccaagagacatcacgtgtgtacacacacacatgcgcgcgcgcgcacacacacacacacacacacacacacacacacacacacacacacgggtgtataggtgagttaaaaggatgtcctcgctgacatcctaattttgtcttaagtttttttggagggagaaaggaaagaggcagggaagatacgtagctctagctttagtcaggcagcctggggggatccccaagcctatgtatggaaccctggtacgaaagcgccctgtgaggagtgggatttcagttttatctgtagaccagatgagaaggagaaaggccccattttgtacatagttgcaacttaaaatttttggcttgcaaaatatttttgtaataaagatttctgggtaac

(19) United States
(12) Patent Application Publication
(10) Pub. No.: US 2005/0138675 A1
(43) Pub. Date: Jun. 23, 2005

(54) Method for determining cardiotoxicity

(75) Inventors: Brad Phillip Hirakawa, San Diego, CA (US); Bart Andrew Jessen, San Diego, CA (US); Gregory J. Stevens, San Diego, CA (US)

Correspondence Address: AGOURON PHARMACEUTICALS, INC., 10350 NORTH TORREY PINES ROAD, LA JOLLA, CA, 92037, US

(73) Assignee: Pfizer Inc.

(21) Appl. No.: 10/980,571

(22) Filed: Nov. 2, 2004

Related U.S. Application Data
(60) Provisional application No. 60/516,744, filed on Nov. 4, 2003.

(51) Int. Cl. A01K 67/027; C12Q 1/68; C12N 9/10

(52) U.S. Cl. 800/3; 435/6

(57) ABSTRACT

The invention relates to methods for characterizing the cardiotoxicity of an agent.

This application claims the benefit of provisional application Ser. No. 60/516,774, filed Nov. 3, 2003, which is incorporated herein by reference in its entirety.

### FIELD OF THE INVENTION

The present invention relates to methods for characterizing the cardiotoxicity of an agent.

### BACKGROUND OF THE INVENTION

Prescription and over the counter medications have an associated dose-dependent toxicity (overdose potential), as well as side effects that can occur at the prescribed dose. While this is fairly common knowledge, the fact that these adverse effects can limit the effectiveness of these medications, may be less familiar. That is, a medication may have the potential to alleviate a disease condition, but the dose necessary to achieve relief produces side effects that are too extensive to warrant the risk of such a dose. These adverse events are still a problem despite extensive measures taken to ensure drug safety (Park, K. B. and Pirmohamed, M. (2001). Toxicology Letters, 120, 281-291). They occur despite extensive preclinical evaluation of drug safety in laboratory animals, as well as clinical trials with large groups of patients.

Cardiotoxicity is one of the adverse events associated with certain chemotherapeutic agents used to treat hematologic and solid malignancies (L'Ecuyer, T., et al. (2001). Molecular Genetics and Metabolism, 74, 370-379). This dose-dependent cardiotoxicity has been a significant complication of the administration of these chemotherapeutic agents, and can occur after acute or cumulative dosing (Boucek, et al. (1999). Journal of Cellular Cardiology, 31, 1435-1446). Cardiotoxicity can also manifest itself with agents given to regulate the heartbeat during conditions where abnormal cardiac rhythms persist (Alvarz-Cedron L., et al. (1998). Bilo. Pharm. Bull, 21(8), 839-843). Clinical effects range from lesions on cardiac tissue, to overt contractile failure.

Currently, these effects are identified through the use of functional tests such as an electrocardiogram (ECG), as well as biochemical biomarkers such as serum troponin measurement.

Until recently, the molecular biology techniques required for identification of biomarkers focused on single or small groups of genes. With the advent of microarray technology, thousands of genes can now be rapidly analyzed. Microarray technology is being increasingly used in the drug discovery process, with applications that include biomarker determination and the associated toxicogenomics (Butte, A. (2002). Nature Reviews, 1, 951-960).

PCT patent application publication WO 97/13877 and related U.S. Pat. No. 6,228,589 disclose methods for assessing the toxicity of a compound in a test organism by measuring gene expression profiles of selected tissues.

U.S. Pat. No. 5,811,231 describes methods and diagnostic kits for identifying and characterizing toxic compounds, wherein the methods and kits measure transcription or translation levels from genes linked to native eukaryotic stress promoters.

There exists a need to identify, characterize and understand the mechanism of action of toxicologically relevant genes in order to simplify the development, screening, and testing of new drug and chemical substances.

### SUMMARY OF THE INVENTION

One aspect of this invention provides a method of characterizing an agent, comprising, treating a mammalian heart cell or a mammal with an agent; and determining the effect of said agent on expression in said mammalian cell or mammal of at least one gene selected from the gene-set of Table 1, wherein said agent is characterized as producing cardiotoxic effects if the agent causes an increase or decrease in expression of at least one gene selected from the gene-set of Table 1.

Another aspect of the invention provides a method of identifying an agent that has cardiotoxic effects comprising
treating a mammalian heart cell or a mammal with an agent; and
determining the effect of said agent on expression in said mammalian cell or mammal of at least one gene selected from the gene-set of Table 1.

Preferably, the determining step comprises determining the effect of said agent on expression of at least two, more preferably, at least three genes, selected from the gene-set. More preferably, the determining step comprises determining the effect of the agent on expression in the mammalian cell or mammal of at least one gene selected from GenBank accession numbers Al176456 metallothionein, X01118 gamma-rANP atrial natriuretic peptide, X89225 L-like neutral amino acid transport protein, J02722 heme oxygenase heat shock protein 32, and AA957003 intercellular calcium-binding protein. Alternatively, more preferably, the determining step comprises determining the effect of the agent on expression in the mammalian cell or mammal of at least one gene selected from GenBank accession numbers Af016296 neuropilin, X52140 integrin alpha-1, Ai638989 unknown, AA899106 cyclin D2, A1170776 GRB2 growth factor receptor bound protein, A1008852 unknown, M892801 unknown, X71127 complement protein C1q beta chain, U14950 synapse-associated protein 97, M57276 leukocyte antigen, AB005743 fatty acid transporter, U68272 interferon gamma receptor, and D85183 SHPS-1.

In a preferred embodiment of the methods of this invention, said mammal cell or mammal is a rat cell or rat, respectively.

The term “gene-set” means the genes listed in Table 1 and their respective homologues and orthologues. The term “homolog” as used herein means a gene having at least 95 percent identity to the applicable gene in Table 1. The term “ortholog” as used herein means a gene of a species other than the species from which the applicable gene in Table 1 is derived, having at least 65 percent identity, preferably 75 percent identity, and even more preferably 85 percent identity, to the applicable gene in Table 1.

The term “cardotoxidty”, “cardiotoxic effects” and similar terms refer to cardiac tissue damage. The damage to the cardiac tissue may include, for example, damage resulting from lesions, contractile failure, hypoxia, myocardial cytotoxicity, histopathological tissue changes, or hemodynamic stress.

“ Increase in expression”, “decrease in expression” and similar terms, when used in reference to the expression of one or more genes, means expression that represents at least a statistically significant increase or decrease as measured against a control. With regard to the present invention, a two-fold change was considered statistically significant, with the proviso that a smaller or larger change in expression may also be statistically significant in some instances.

“Nucleotide identity” as used herein refers to the sequence alignment of a nucleotide sequence calculated against another nucleotide sequence. Specifically, the term refers to the percentage of residue matches between at least two nucleotide sequences aligned using a standardized algorithm. Such an algorithm may insert gaps in the sequences being compared in a standardized and reproducible manner in order to optimize alignment between the sequences, thereby achieving a more meaningful comparison. Nucleotide identity between nucleotide sequences is preferably determined using the default parameters of the CLUSTAL W algorithm as incorporated into the version 5 of the MEGALIGN sequence alignment program. This program is part of the LASERGENE suite of molecular biological analysis programs (DNASTAR, Madison Wis.). CLUSTAL W is described in Thompson, J. D., et al. (1994) Nucleic Acids Research 22, 4673-4680.

“Nucleotide sequence” and “polynucleotide” refer to both DNA or RNA of genomic or synthetic origin which may be single-stranded or double-stranded and may represent a sense or an antisense strand. The term “complementary nucleotide sequence” refers to a nucleotide sequence that anneals (binds) to another nucleotide sequence according to the pairing of a guanidine nucleotide (G) with a cytidine nucleotide (C), and an adenosine nucleotide (A) with a thymidine nucleotide (T), except in RNA where a T is replaced with a uridine nucleotide (U) so that U binds with A.

The agent can be any type of chemical compound. Generally, the agent is a xenobiotic compound or a pharmacological compound.

### BRIEF DESCRIPTION OF THE DRAWINGS

FIG. 1 shows a Venn diagram displaying only those genes whose expression levels increased by ≧2 fold with respect to controls at the 6 hour time-point. Diagrams represent genes taken from all genes on the RG U34A array.

FIG. 2 shows a Venn diagram displaying only those genes whose expression levels increased by ≧2 fold with respect to controls at the 24 hour time-point. Diagrams represent genes taken from all genes on the RG U34A array.

### DETAILED DESCRIPTION OF THE INVENTION

An alternative to current biomarkers are gene expression biomarkers of cardiotoxicity. Such biomarkers reflect changes induced at the gene expression level that are indicative of adverse cardiac effects. A gene expression biomarker has the potential to identify these cardiac effects at an earlier time, and can therefore be beneficial in limiting or preventing subsequent cardiac tissue damage. During the drug development process, gene expression biomarkers can shorten the length of preclinical studies by reducing the need for studies that require long-term endpoints. For example, if a gene expression biomarker of compound-induced cardiotoxicity that has been shown to precede cardiac lesions is found early, the study can then be ended without the need to wait the time required for the development of cardiac lesions.

Elucidation of a gene expression biomarker of cardiac toxicity that is specific to a given compound or condition is valuable for specific cases or mechanistic studies. However, a biomarker of gene expression that reflects a more general condition of cardiac stress would be more desirable for the preclinical or clinical setting where the question would simply be whether or not the heart has been adversely affected. The process of identifying gene expression biomarkers of cardiac damage was begun by generating lists of genes that are affected by various compounds or conditions that are known to cause cardiac tissue damage. Analysis of these gene lists revealed genes that are measurably affected by a range of cardiac stressors, and therefore are useful as biomarkers of cardiotoxicity or cardiac stress.

Accordingly, one embodiment of the present invention provides methods using the genes listed in Table 1 for identifying agents that are cardiotoxic as a result of administration of the agent to the mammalian cells or mammal.

It will be appreciated by those with skill in the art based upon the present disclosure, that any mammalian cardiac tissue or heart cell (collectively referred to as heart cell) may be used in the practice of this invention. Mammalian heart cell lines that propagate indefinitely are preferred. Cardiac tissue or heart cells may be derived from any mammal, but preferably are derived from mouse, rat, dog or human, more preferably rat, mouse or human. As described below, it is preferable to match the species from which the cells are derived to the species of the gene fragments used as probes or microarray oligomers in detecting expression of the genes herein described.

Any mammal may be used in the practice of this invention. Preferably, such mammal is a rodent, more preferably a rat or mouse, even more preferably, a rat. As described below, it is preferable to match the species of mammal to the species of the gene fragments used as probes or microarray oligomers in detecting expression of the genes herein described.

It will be appreciated by those with skill in the art based upon the present disclosure that, in the practice of the invention, the determination of gene expression of the genes described herein in a mammal is performed, following treatment of the mammal with a test agent, on cardiac tissue or cells.

In the method aspects of this invention, the effect of a test agent on expression of toxicologically relevant genes is analyzed. The detection of changes in gene expression is preferably performed by measuring messenger RNA (mRNA) expression of a gene by methods well known to those with skill in the art based upon the present disclosure. For example, one method that may be employed to measure mRNA expression involves polymerase chain reaction (PCR) and gel electrophoresis to detect differentially expressed genes. For example, the product from PCR synthesis may be subjected to gel electrophoresis, and bands produced by two or more mRNA populations may be compared. Bands present on an autoradiograph of one gel from one mRNA population, and not present on another, correspond to the presence of a particular mRNA in one population and not in the other, and thus indicate a gene that is likely to be differentially expressed. (See, Williams, J. G. (1990) NucL Acids Res. 18, 6531; Welsh, J., et al., (1990) NucL Acids Res., 18, 7213; Woodward, S. R., (1992) Mamm. Genome, 3, 73; and Nadeau, J. H. (1992) Mamm. Genome 3, 55; Liang, P. et al., (1992) Science 257, 967; Welsh, J. et al. (1992) Nucl. Acid Res. 20, 4965; Liang, P., et al. (1993) Nucl. Acids Res. 3269, 1993; and U.S. Pat. Nos. 6,114,114 and 6,228,589).

As used herein, the terms “arrays” and “microarrays” refer to an array of distinct polynucleotides or oligonucleotides synthesized on a substrate, such as paper, nylon or other type of membrane, filter, chip, glass slide, or any other suitable solid support. In a preferred embodiment, microarrays may be prepared and used according to the methods described in U.S. Pat. No. 5,837,832, PCT application WO95/11995, Lockhart et al. (1996) Nat. Biotech. 14:1675-1680 and Schena, M. et al. (1996) Proc. Natl. Acad. Sci._{—}93:10614-10619, all of which are incorporated herein in their entirety by reference. In other embodiments, such arrays are produced by the methods described by Brown et al., U.S. Pat. No. 5,807,522.

A preferred method for detecting mRNA expression is by using microarrays. The process of isolating mRNA from cells or tissues exposed to a stimulus (e.g., drugs or chemicals) and analyzing the expression with gel electrophoresis can be laborious and tedious. To that end, microarray technology provides a faster and-more efficient method of detecting differential gene expression. Differential gene expression analysis by microarrays involves nucleotides immobilized on a substrate whereby nucleotides from cells that have been exposed to a stimulus can be contacted with the immobilized nucleotides to generate a hybridization pattern. See, for example, Diehn M, et al. (1993) Nat Genet 25(1), 58-62; Scherf, U., et al. Nat Genet 24(3): 236-44 (1993); Hayward R. E., et al. (1993) Mol. Microbiol. 35(1), 6-14; Johannes G., et al. (1993) Proc. Natl. Acad. Sci. USA 96(23), 13118-23.

A microarray is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray or detection kit, it may be preferable to use oligonucleotides that are only 7-20 nucleotides in length. The microarray or detection kit may contain oligonucleotides that cover the known 5′, or 3′, sequence, sequential oligonucleotides which cover the full length sequence; or unique oligonucleotides selected from particular areas along the length of the sequence. Polynucleotides used in the microarray or detection kit may be oligonucleotides that are specific to a gene or genes of interest.

To produce oligonucleotides to a known sequence for a microarray or detection kit, the genes of interest are typically examined using a computer algorithm which starts at the 5′ or at the 3′ end of the nucleotide sequence. Typical algorithms may then be used to identify oligomers of defined length that are unique to the gene, have a GC content within a range suitable for hybridization, and lack predicted secondary structure that may interfere with hybridization. In certain situations it may be appropriate to use pairs of oligonucleotides on a microarray or detection kit. The “pairs” are identical, with the exception of one nucleotide, preferably located in the center of the sequence. The second oligonucleotide in the pair (mismatched by one) serves as a control. The number of oligonucleotide pairs may range from two to one million. The oligomers are synthesized at designated areas on a substrate using a light-directed chemical process. The substrate may be paper, nylon or other type of membrane, filter, chip, glass slide or any other suitable solid support.

An oligonucleotide may be synthesized on the surface of the substrate by using a chemical coupling procedure and an ink jet application apparatus, as described in PCT application WO95/251116. A “gridded” array analogous to a dot (or slot) blot may be used to arrange and link cDNA fragments or oligonucleotides to the surface of a substrate using a vacuum system, thermal, LTV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number between two and one million which lends itself to the efficient use of commercially available instrumentation.

As will be appreciated by those with skill in the art, it is preferable to match the species from which the expression samples are derived to the species of the gene fragments oligomers used in the preparation of microarrays. For example, if expression samples are prepared from a human cell line, then a microarray containing human gene fragments is preferably used containing the applicable gene from the gene-set in Table 1 or its applicable ortholog.

To conduct sample analysis using a microarray, hybridization probes are prepared from the RNA of the biological sample to be tested. For example, the mRNA may be isolated, and cDNA produced and used as a template to make antisense RNA (aRNA). The aRNA may then be labeled by amplifying in the presence of labeled nucleotides (e.g., fluorescently labeled nucleotides). Labeled probes are incubated with the microarray so that the probe sequences hybridize to complementary oligonucleotides of the microarray. Incubation conditions are adjusted so that hybridization occurs with precise complementary matches or with various degrees of less complementary matching.

After removal of non-hybridized probes, a scanner may be used to determine the levels and patterns of fluorescence. The scanned image may then be examined to determine the degree of complementarity and the relative abundance of each oligonucleotide sequence on the microarray.

In a preferred embodiment of the invention, microarrays are used to detect the expression of the genes listed in Table 1. The method comprises incubating a test sample with one or more nucleic acid molecules and assaying for binding of the nucleic acid molecule with components within the test sample. Such assays will typically involve arrays comprising many genes, at least one of which is a gene listed in Table 1 of the present invention.

Quantitative real time PCR techniques (Q-PCR) are used to further analyze the genes of interest and are applied to each individual sample, rather than the pooled sample analysis used with the GeneChips. This approach provided measurements of variability and statistics. Individual RNA samples are reverse transcribed using a poly dT oligonucleotide primer to produce single stranded cDNA. The Primers used, listed in Table 2, were selected using on-line software that optimizes primer selection for product size and favorable Q-PCR reaction conditions (http://frodo.wi.mit.edu/cgi-bin/primer3/primer3_www.cgi). The target sequence was PCR amplified by combining primer pairs in a solution of PCR Master Mix (Promega, San Luis Obispo Calif.). PCR fragments of expected size (as determined by gel electrophoresis) were cloned using a TOPO TA cloning kit (Invitrogen, Carlsbad, Calif.) with subsequent plasmid DNA purification using a OlAprep Spin Miniprep Kit (Qiagen, Valencia, Calif.). Plasmid DNA, linearized with Bgl II, was then used to generate standard curves for subsequent sample analysis on a LightCycler thermocycler (Roche Diagnostics Corp., Indianapolis, Ind.). Single stranded cDNA or plasmid standards were amplified for quantitation using the Quantitect™ SYBR Green PCR kit (Qiagen). Q-PCR conditions for sample analysis involved an initial denaturation at 95° C. for 900 sec, followed by 50 cycles of amplification. Each cycle included denaturation at 94° C. for 15 sec, oligonucleotide annealing at 50° C. for 20 sec, product synthesis at 72° C. for 30 sec., and quantitative acquisition at 1-2° C. below the melting temperature of the product. Table 3 lists the biomarkers confirmed by Q-PCR.

Conditions for incubating a nucleic acid molecule with a test sample vary. Incubation conditions depend on the format employed in the assay, the detection methods employed and the type and nature of the nucleic acid molecule used in the assay. One skilled in the art will recognize that any one of the commonly available hybridization, amplification or array assay formats can readily be adapted to employ fragments of the genes listed in Table 1. Examples of such assays can be found in Chard, T (1986), An Introduction to Radioimmunoassay and Related Techniques, Elsevier Science Publishers, Amsterdam, The Netherlands; Bullock, G. R. et al. Techniques in Immunocytochemistry, Academic Press, Orlando, Fla. Vol. 1 (1982), Vol-2 (1983), Vol-3 (1985); Tijssen, P. (1985) Practice and Theory of Enzyme Immunoassays: Laboratory Techniques in Biochemistry and Molecular Biology, Elsevier Science Publishers, Amsterdam, The Netherlands.

Methods for preparing nucleic acid extracts of cells in the practice of the present invention are well known in the art and can be readily adapted in order to obtain a sample that is compatible with the system utilized.

TABLE 1

| Genbank # | DIG | DOX | ISO | LPS | common name | biological function |
|---|---|---|---|---|---|---|
| | 6 hour | | | | | |
| AI176456 | up | up | up | up | metallothionein | oxidative stress |
| X01118 | down | down | up | down | gamma-rANP | blood pressure |
| X89225 | up | up | up | up | L-like neutral amino acid transport protein | amino acid transport |
| J02722 | up | up | up | up | heme oxygenase (hsp32) | heat shock protein |
| AA957003 | up | up | up | up | intercellular calcium-binding protein | signal transduction |
| | 24 hour | | | | | |
| AF016296 | down | down | down | down | neuropilin | vasculogenesis |
| X52140 | down | down | down | down | integrin alpha-1 | ECM/signal transduction |
| AI638989 | down | down | down | down | EST | unknown |
| AA899106 | down | down | down | down | cyclin D2 | cell cycle proliferation |
| AI170776 | up | up | up | up | GRB2 | hypertrophy |
| AI008852 | up | up | up | up | EST | unknown |
| AA892801 | up | up | up | up | EST | unknown |
| X71127 | up | up | up | up | complement protein C1q beta chain | immune response |
| U14950 | up | up | up | up | synapse-associated protein 97 | unknown |
| M57276 | up | up | up | up | leukocyte antigen MRC-OX44 | unknown |
| AB005743 | up | up | up | up | fatty acid transporter | metabolism |
| U68272 | up | up | up | up | interferon gamma receptor | immune response |
| D85183 | up | up | up | up | SHPS-1 | unknown |

TABLE 2

| | | | |
|---|---|---|---|
| Genbank# | Common Name | Left Primer | Right Primer |

| | | | |
|---|---|---|---|
| AI176456 | metallothionein 2 | acctcagcctgacttctacc | tggataacgttggatgactc |
| X01118 | natriuretic peptide precursor type A | tagtgaggccttacctctcc | cgtggtgctgaagtttattc |
| X89225 | L-like neutral aa transport protein | aactataagggccagaatgc | gtgatattctgccactcagc |
| J02722 | heme oxygenase 1 | tgtgccttgatacactaccc | tcagtttcagtcacccactc |
| AA957003 | S100 calcium-binding protein (calgranulin A) | taccgaaagcttgttcaaag | gtggctgtctttatgagctg |
| AF016296 | neuropilin | gctctcacaagacattctgc | attcctctggcttctggtag |
| X52140 | integrin alpha 1 | gcatttcagacctcactctg | atcagatttggtgaatgctg |
| AA899106 | cyclin d2 | agggaaaggccatatagttg | cactagttcccaagcacaag |
| AI170776 | growth factor receptor bound protein 2 | acaagaattacacccacacg | ggcatcagctggactatatg |
| AI008852 | eukaryotic translation elongation factor 1a1 | actaaggagggtgctctttg | tcaggacactgaatctccac |
| AA892801 | eukaryotic translation elongation factor 2 | ttctcaaacctggtatggtg | cacgttctttacgttgaagc |
| X71127 | complement component 1, | tagtcttgaagctggagcag | ggaggttttggataggagtg |
| U14950 | synapse associated protein | caactagaccaaagcgtgac | ggatagagctgtgcaatctg |
| M57276 | CD53 antigen | gaaaatttggagtcccattc | ggcaatgtaattcaaagctg |
| AB005743 | Cd36 | attactggagccgttattgg | aaagcaatggttccttcttc |
| U68272 | interferon gamma receptor | tcaatgtgagtcaggaaacc | taacttgccagaaagacgac |
| D85183 | Protein tyrosine phosphatase, (SHP substrate 1) | ctgaggtaatcagtgcaagg | tagcgcctcaacttactgag |

TABLE 3

| | DIG | | DOX | | ISO | | LPS | |
|---|---|---|---|---|---|---|---|---|
| Genbank # | Array | Q-PCR ± SD | Array | Q-PCR ± SD | Array | Q-PCR ± SD | Array | Q-PCR ± SD |
| | 6 hour | | | | | | | |
| AI176456 | 3.43* | 2.88* ± 1.96 | 2.16 | 2.55* ± 0.20 | 126.27* | 12.05* ± 9.77 | 327.72* | 101.44* ± 56.50 |
| X01118 | 0.38* | 0.40* ± 0.65 | 0.47* | 0.12* ± 0.08 | 2.32* | 2.30* ± 0.63 | 0.33* | 0.27* ± 0.08 |
| X89225 | 2.18 | 6.04* ± 3.28 | 2.29* | 5.49* ± 3.10 | 3.04* | 2.51* ± 1.27 | 2.44* | 8.86* ± 6.01 |
| J02722 | 2.47* | 3.06* ± 0.48 | 2.27* | 3.74* ± 2.72 | 13.61* | 5.31* ± 2.69 | 14.69* | 2.91* ± 0.31 |
| AA957003 | 4.33* | 7.71* ± 7.08 | 2.24* | 17.99* ± 12.20 | 3.90* | 15.61* ± 10.48 | 4.00* | 8.86 ± 6.16 |
| | 24 hour | | | | | | | |
| AF016296 | 0.48* | 0.35* ± 0.41 | 0.42* | 0.21* ± 0.04 | 0.44* | 0.34* ± 0.17 | 0.28* | 0.27* ± 0.38 |
| X52140 | 0.23* | 0.49 ± 0.10 | 0.37* | 0.30 ± 0.23 | 0.42* | 0.27* ± 0.03 | 0.17* | 0.37* ± 0.09 |
| AA899106 | 0.20* | 0.23* ± 0.10 | 0.32* | 0.37* ± 0.15 | 0.23* | 0.39 ± 0.18 | 0.06* | 0.25* ± 0.19 |
| AI170776 | 2.94* | 19.15* ± 18.9 | 2.01* | 35.72* ± 14.26 | 2.70* | 11.30* ± 10.33 | 2.18* | 12.64* ± 7.97 |
| AI008852 | 2.23* | 3.04* ± 1.66 | 2.01* | 3.64* ± 2.43 | 10.4* | 10.46* ± 4.02 | 3.47* | 5.77* ± 2.89 |
| AA892801 | 2.46* | 2.69* ± 1.48 | 2.41* | 2.07* ± 0.90 | 8.14* | 7.92* ± 2.20 | 2.58* | 4.41 ± 2.03 |
| X71127 | 2.22* | 36.04* ± 16.29 | 5.04* | 42.99* ± 31.59 | 3.01* | 33.81* ± 10.93 | 3.70* | 96.43* ± 54.67 |
| U14950 | 3.83* | 13.83* ± 12.98 | 2.02 | 46.89* ± 28.79 | 2.05 | 18.35* ± 9.62 | 2.19 | 66.29* ± 67.06 |
| M57276 | 3.79* | 8.53 ± 8.18 | 2.88 | 6.04 ± 5.67 | 2.81* | 7.83* ± 6.56 | 4.17* | 7.40* ± 4.97 |
| AB005743 | 2.84* | 2.34* ± 2.54 | 3.57* | 8.52 ± 6.16 | 2.69* | 8.78* ± 7.90 | 2.15* | 5.58* ± 3.80 |
| U68272 | 2.25* | 23.17* ± 9.32 | 2.61* | 46.3* ± 44.50 | 17.1* | 19.06* ± 6.24 | 2.96* | 19.32* ± 11.81 |
| D85183 | 3.00* | 5.72* ± 4.78 | 3.02 | 8.54* ± 6.01 | 10.31* | 7.11 ± 6.79 | 3.70* | 7.27* ± 5.02 |
| Fold Change Values: | | | | | | | | |
| *= Affymetrix, significant change value | | | | | | | | |
| *= Q PCR, t-test, p < 0.05 | | | | | | | | |
| Genes induced or suppressed at least 2-fold relative to vehicle control levels, based on requirement that genes are statistically present in all samples within each time point. RT-PCR samples were completed on individual samples (n = 4/group) for subsequent statistical analysis (students t-test) and measurement of variability (standard deviation). Affymetrix | | | | | | | | |
| # MAS software was used to generate statistical change values for pooled (n = 4 pooled samples/group) samples. | | | | | | | | |

To locate potential biomarkers of cardiotoxicity, gene expression data were mined for common genes that are affected by all four xenobiotics, digoxin, doxorubicin, isoproterenol, and lipopolysaccharide, with the hopes of identifying a subset of genes that could be considered cardiotoxic predictors. A fold change criteria was used, which can be used to increase the stringency of these types of comparisons. In this manner, gene lists were generated that include genes that have been up or down-regulated relative to controls, at a level to be determined by the researcher. At the current stage in the evolution of this type of gene expression data, fold-change cut off values seem to vary between researchers (Butte, A. (2002). Nature Reviews, 1, 951-960). That is, there are no steadfast rules that govern the analysis criteria. Intuitively selected fold-change values will depend on factors such as specific xenobiotic, species, test condition, or fold-change values that are deemed biologically relevant from either previous experimental data or robust hypotheses. With regard to the present invention, a fold change of two was used to filter data, with the assumption that fold changes less than two can be considered not biologically relevant or within the “noise” of the experiment. However it is important to remember that for some genes, a small increase in expression may play a significant biological role. Inversely, some may require much more than a 2-fold change to produce a biological effect.

Metallothionein (MT) gene expression was induced in cardiac ventricular tissue by all four compounds six hours after acute systemic administration. MT is commonly known for its role in detoxification of heavy metals (Templeton, D. M. and Cherian, M. G. (1991). Methods in Enzymology, 205, 11-24). However, the idea that this is its primary role does not agree with evolutionary trends. That is, the structure of MT appears to be highly conserved across species, and therefore it likely that MT performs an evolutionary conserved role and not a function that helps an organism in the event of exposure to recent environmental toxicants (Kang, Y. J., et al. (1997). Journal Clin Invest, 100, 1501-1506; Palmiter, R. D. (1998). Proc Natl Acad Sci, 95, 8428-8430; Valle, B. L. (1995). The function of metallothionein. Neurochem Int, 27, 23-33). Perhaps more appropriately, MT has also been implicated as a free radical scavenger (Thornalley, P. J. and Vasak, M. (1985). Biochim Biophys Acta, 827, 236-44). Accordingly, MT gene expression is up-regulated under several experimentally induced pro-oxidant conditions (Sato, M. and Bremner, I. (1993). Free Raic Biol Med, 14, 325-337). Additionally, myocardial oxidative injury has been shown to be involved in conditions such as exposure to environmental chemicals and therapeutic drugs such as DOX, as well as ischemia-reperfusion injury, with transgenic MT over-expressing animals showing marked resistance to such insults, and co-treatment with supplemental MT compounds producing similar protective results (Kang, Y. J., et al. (1997). Journal Clin Invest, 100, 1501-1506; Satoh, M. (1988). Toxicology, 53, 231-237). MT is believed to protect against myocardial oxidative injury though mechanisms that involve direct reactivity with reactive oxygen species, as well as mechanisms that involve the mobilization of zinc from MT to be used in cellular defense mechanisms against oxidative stress (Thomas, J. P., et al. (1986) Biochim Biophys Acta, 884, 448-461; Sato, M. and Bremner, I. (1993). Free Raic Biol Med, 14, 325-337).

Complement protein gene expression was induced in myocardial tissue by all four compounds at the 24-hour time point. Previous studies have shown that complement proteins gene expression is induced following myocardial tissue injury (Koji, Y., et al. (1998). Circ Res, 83, 860-869). Also, recent evidence implicates complement proteins as inflammatory mediators that are involved in the pathogenesis of various heart diseases, which eventually lead to the decline of heart function (Afenasyeva, M. and Rose, N. R. (2002). American Journal of Pathology, 161(2), 351-357; Koji, Y., et al. (1998). Circ Res, 83, 860-869). Complement (also called alexin) represents a complex system of over 30 proteins (Baldwin III, W. M., et al. (2002). Current Opin Organ Transplant, 7, 92-99). These inflammatory mediators can affect cardiomyocyte homeostasis and thus activate signaling cascades, which can result in deleterious effects on myocardial function.

Heme oxygenase gene expression was induced in myocardial tissue by all four compounds at the 6-hour time point. Heme oxygenase catalyzes the breakdown of heme to the antioxidant bilirubin and the vasodilator carbon monoxide. Recent research also categorizes heme oxygenase as a heat shock protein involved in cardiovascular protection against oxidative stress, that functions as a molecular chaperone to protect against cellular stress or insult (Vulapalli, S. R., et al. (1999). Journal of Molecular and Cellular Cardiology, 31(8), 1581-1589). Aortic heme oxygenase gene expression and subsequent protein levels have been shown to increase in animal models of hypertension (Nobukazo, I., et al. (1997). Circulation, 96, 1923-1929). Additionally, heme oxygenase gene expression is induced by oxidative and hemodynamic stress to the heart, particularly after ischemic insults (Sharma, H. S., et al. (1996). Mol Cell Biochem, 157, 111-116; Maulik, N., et al. (1996). Journal Mol Cell Cardiol, 28, 1261-1270).

Neuropilin gene expression was induced in myocardial tissue by all four compounds at the 24-hour time point. It has been demonstrated that tissue hypoxia induces the expression of hypoxia-induced factor (HIF-1 alpha), and subsequently activates expression of vascular endothelial growth factor (VEGF) and its receptors fit-1 neuropilin-1, and angiopoietin-2 (Ang-2) (Semenza, G. L. (2000). Journal Appl Physiol, 88, 1474-1480). Additionally, several genes related to angiogenesis are induced after myocardial ischemia and coronary occlusion (Banai, S., et al. (1994). Cardiovasc Res, 28, 1176-1179; Deindl, E. and Schaper, W. (1998). Mol Cell Biochem, 186, 43-51.)

Atrial natriuretic peptide (ANP) is produced mainly in the atria and is released into plasma in patients with increased intravascular volume such as in the case of heart failure. Very little ANP is found in the plasma of healthy individuals, but is found in hypertrophied and failing ventricular tissue (Saito, Y., et al. (1989). Journal Clin Invest, 125, 298-305). ANP promotes natriuresis, increases venous capacitance, reduces sympathetic tone by dampening baroreceptors, suppresses catecholamine release from autonomic nerve endings, and suppresses sympathetic outflow from the central nervous system (Hunt, P. J., et al. (1996). J Clin Endocrinol Metab, 81, 3871-3876). Additionally, ANP suppresses reflex tachycardia and vasoconstriction to facilitate the decrease of mean arterial pressure in the case of increased cardiac preload. Plasma ANP concentrations have been shown to correlate with severity of heart failure (Hara, H., et al. (1987). Clin Cardiol, 10, 437-442), and has been shown to be a predictor of long-term mortality and morbidity in myocardial infarction patients (Hall, C., at al. (1994). Circulation, 89, 1934-1942). ANP levels also increase in plasma of patients with cardiac conditions (Tulevski, I. I., et al. (2001). Heart, 86, 27-30).

Additional genes affected by all compounds do not yet have clearly identified roles in cardiac tissue damage, although initial data are available. Synapse-associated protein 97 (SAP97) is co-localized in the T-tubules of cardiac ventricular myocytes, with a possible role in macromolecular signaling complexes and inward rectifier potassium channels (Kir2.2) in the heart (Leonoudakis, D., et al. (2000). Journal of Cell Science, 114, 987-998). Also, interferon gamma may function as a regulatory cytokine early in the pathogenesis of myocardial inflammation (Smith, S. C. and Allen, P. M. (1992). Circulation Research, 70, 856-863; Eriksson, U., et al. (2001). Journal of Immunology, 167, 5464-5469). Extracellular matrix remodeling occurs in the cardiac myocyte hypertrophic response, and integrins appear to be involved (Pham, C. G., et al. (2000). Am J Physiol heat Circ Physiol, 279, H2916-H2926). Integrins mediate cell-matrix adhesion and certain forms are expressed exclusively in the cardiac and skeletal muscle. At the moment, it is clear that integrins are involved in important adhesive and signaling functions necessary for the repair of compromised myocardial tissue, however more specific modes of action are still unknown (Nawata, J., et al. (1999). Cardiovasc Res, 43, 371-381; Burgess, M. L., et al. (1994). Cir Res, 74, 291-298; Ross, R. S., Borg, T. K. (2001). Circ Res, 88, 1112-1119). Additionally, growth factor receptor bound protein-2 (Grb2) has been associated with integrin mechanisms and cardiac hypertrophy in the event of certain insults (Zhang, S., et al. (2003). Journal Clin Invest, 11, 833-841). Other genes identified in these experiments are certainly in involved with metabolism (fatty acid transporter, amino acid transporter), cell cycle proliferation (cyclin), signal transduction (intercellular calcium-binding protein), in various tissues, but the their role in the myocardium are not currently understood.

The disclosures of all patents, applications, publications and documents, for example brochures or technical bulletins, cited herein, are hereby expressly incorporated by reference in their entirety.

It is believed that one skilled in the art can, using the present description, including the examples, drawings, sequence listings and attendant claims, utilize the present invention to its fullest extent. The following Examples are to be construed as merely illustrative of the practice of the invention and not limitative of the remainder of the disclosure in any manner whatsoever.

### EXAMPLES

### Example 1

### Effects of Digoxin, Doxorubicin, Isoproterenol and Lipopolysaccharide on Cardiotoxicity

Materials and Methods:

Male Sprague-Dawley rats weighing between 175-220 grams were used for all studies. Animals were purchased from Charles River Laboratories (Wilmington, Mass.) and allowed to acclimate for one week prior to use. All animals were given food and water ad libitum, and housed under a 12-hour light/12-hour dark cycle. The animals were housed in conventional plastic bottom cages (2 animals/cage) with corncob bedding. All dosing with test compounds was a single acute injection by the intraperitoneal route, in normal saline, with a 20×¾″ disposable needle attached to a 1 cc disposable syringe. These animals were euthanized by a 150 mg/kg intraperitoneal injection of Nembutal prior to collection of heart tissue.

Dose range-finding studies were completed to determine a xenobiotic dose that would produce signs of cardiotoxicity such as elevated serum troponins, creatine kinase isoenzymes, or histo-pathological tissue changes, without causing overt general toxicity. Comprehensive literature reviews were also used to select doses. Based on the results of these studies, animals were randomly divided into ten groups (n=6) and given a single intraperitoneal injection of either normal saline vehicle (VEH), 20 mg/kg of digoxin (DIG), 30 mg/kg of doxorubicin (DOX), 70 mg/kg of isoproterenol (ISO), or 10 mg/kg of lipopolysaccharide (LPS). Six or twenty-four hours later, animals were euthanized with a lethal intraperitoneal dose of Nembutal solution. Biopsy punches were used to remove approximately 100 grams of ventricular tissue for subsequent RNA isolation and gene expression analysis. Total RNA was isolated from the samples using a Qiagen RNAeasy protocol (Valencia, Calif.). In this procedure, RNA is captured on a solid phase column, then washed with a series of solutions to purify the sample. In the last step, RNA is eluted from the column with RNAse free water. RNA quality and quantity was then checked with an optical density reading at 260/280 wavelengths, and an aliquot of RNA was run on an RNA denaturing gel. High quality RNA should have an OD 260/280 ratio of 2, and the denaturing gel should show two distinct ribosomal RNA bands.

Synthesis of double stranded cDNA from the total RNA isolated was the first step, which involves the incorporation of a T7-(dT)₂₄ primer into the sequence, with subsequent second strand synthesis which yields a double stranded template for the in vitro transcription (IVT) reaction. The resulting cDNA was run in an IVT reaction using an Enzo BioArray High Yield RNA transcript labeling kit (Enzo Life Sciences, Inc. Farmingdale, N.Y.), during which a biotin label was incorporated into the complementary RNA (cRNA) sequence. This step also amplified the product by generating approximately 40 transcripts per cDNA substrate. After the IVT reaction, cRNA was fragmented by metallic fragmentation, and a small portion of the sample was run on a 1% agarose gel in order to visualize the fragmentation pattern. The range should be between 35 to 200 bases. Four samples from each group showing the highest purity and quality results were pooled for hybridization onto one Genechip to greatly reduce experimental costs. Genes of interest that are found through the use of GeneChip technology, will then be verified using real time PCR techniques on each individual sample.

Next, the cRNA was hybridized to the RG U34A GeneChip oligonucleotide array (Affymetrix Inc, Santa Clara, Calif.). An aliquot of the fragmented cRNA was mixed with control components, injected into the array, and the filled array was allowed to incubate at 45° C. for 16 hours in an Affymetrix oven, which rotates the samples continuously during incubation. The next morning, the array was taken from the oven and the sample was removed and frozen at −80° C. for possible future use. That is, in the event of a defective chip, the hybridization solution can be re-used. The array chamber was then filled with a wash buffer, and placed in an automated fluid exchange system. The fluidics station controls the washing and staining of the probe array with the fluorescently labeled streptavidin stain solution and an antibody solution. After the staining process was complete the chips were scanned by an Agilent GeneArray laser scanner (Agilent, Palo Alto, Calif.).

Raw data was obtained in the form of intensity data, represented by an image of each chip with brighter spots indicating a higher signal for that particular probe set. The Affymetrix MicroArray Suite (MAS) software (version 5.0) (Affymetrix, Santa, Clara, Calif.) generates this data. A statistical algorithm, which takes into account background intensity levels, is used classify gene expression as present or absent. This indicates whether or not the selected gene is present or not present within a given sample. Relative expression levels were also calculated in this manner, using normalizations that correct for varying signal strengths between chips. Once expression values have been generated, other software packages can be applied, such as GeneSpring 4.1 (Silicone Genetics, Santa Clara, Calif.). GeneSpring incorporates the Affymetrix data output files and then normalizes data to median chip expression values in order to allow for across chip comparison. In this manner, comparisons between sample types can be made using Venn diagrams, gene lists, dendrograms, and correlation statistics.

Venn diagrams are made up of two or more overlapping circles and are often used to graphically depict relationships between sets of data. The software allows the quick creation of gene lists from these diagrams.

Results:

Acute in vivo DIG administration (20.0 mg/kg, i.p.) induced and/or suppressed several genes in cardiac ventricular tissue, when compared to the cardiac ventricular tissue of vehicle treated animals. Specifically, 44 genes were expressed in cardiac tissue at least 2-fold higher than those at normal expression levels in vehicle treated animals, 6 hours after treatment and 223 genes 24 hours after treatment. Similarly, 129 genes were suppressed at least 2-fold lower than vehicle treated animal cardiac tissue 6 hours after treatment and 184 genes 24 hours after treatment. Acute in vivo DOX administration (30.0 mg/kg, i.p.) caused the induction of 148 cardiac tissue genes at least 2-fold higher than controls 6 hours after treatment and 231 genes 24 hours after treatment, and suppressed 154 genes at least 2-fold lower than control expression levels 6 hours after treatment and 215 genes 24 hours after treatment. Acute in vivo ISO administration (70.0 mg/kg, i.p.) caused the induction of 330 cardiac tissue genes at least 2-fold higher than controls 6 hours after treatment and 480 genes 24 hours after treatment, and suppressed 98 genes at least 2-fold lower than control expression levels 6 hours after treatment and 175 genes 24 hours after treatment. Acute in vivo LPS administration (10.0 mg/kg, i.p.) caused the induction of 374 cardiac tissue genes at least 2-fold higher than controls 6 hours after treatment and 279 genes 24 hours after treatment, and suppressed 663 genes at least 2-fold lower than control expression levels 6 hours after treatment and 172 genes 24 hours after treatment.

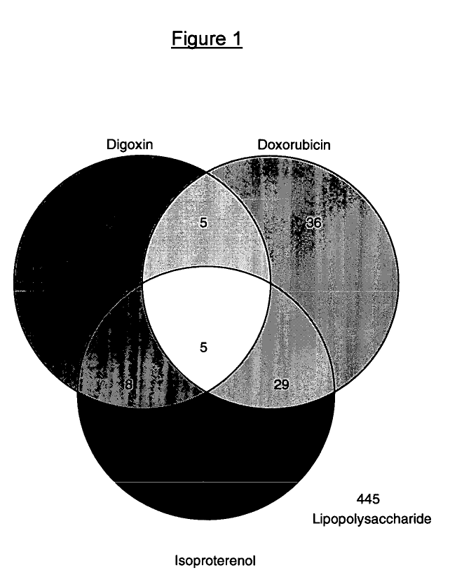

A Venn diagram was generated that encompassed all of the gene lists obtained at the 6-hour time-point (FIG. 1). Each field of the Venn diagram includes all genes either induced or suppressed at least 2-fold relative to control levels. The middle field indicates genes in common to all four compounds, and therefore possible biomarkers of cardiac toxicity. This field revealed the five genes (GenBank accession number followed by common name); Al176456 metallothionein, X01118 gamma-rANP atrial natriuretic peptide, X89225 L-like neutral amino acid transport protein, J02722 heme oxygenase heat shock protein 32, and M957003 intercellular calcium-binding protein (Table 1).

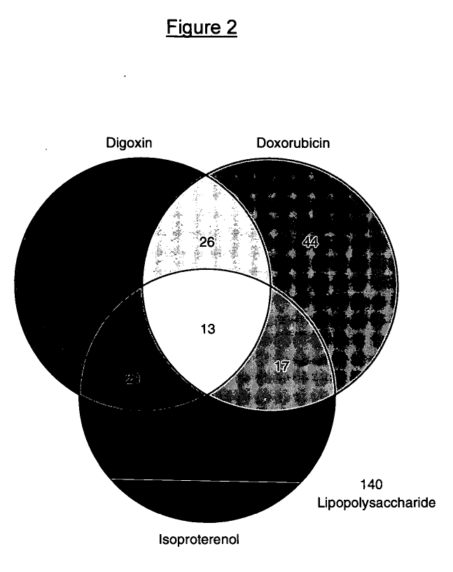

Next, a Venn diagram was generated that encompassed all of the gene lists obtained at the 24-hour time-point (FIG. 2). Each field of the Venn diagram includes all genes either induced or suppressed at least 2-fold relative to control levels. The middle field indicates genes in common to all four compounds, and therefore possible biomarkers of cardiac toxicity. This field revealed 13 genes: (GenBank accession number followed by common name) Af016296 neuropilin, X52140 integrin alpha-1, Ai638989 unknown, AA899106 cyclin D2, Al170776 GRB2 growth factor receptor bound protein, A1008852 unknown, AA892801 unknown, X71127 complement protein C1q beta chain, U14950 synapse-associated protein 97, M57276 leukocyte antigen, AB005743 fatty acid transporter, U68272 interferon gamma receptor, and D85183 SHPS-1 (Table 1).

1. A method of characterizing an agent, comprising, treating a mammalian heart cell or a mammal with an agent; and determining the effect of said agent on expression in said mammalian cell or mammal of at least one gene selected from the gene-set of Table 1, wherein said agent is characterized as producing cardiotoxic effects if the agent causes an increase or decrease in expression of at least one gene selected from the gene-set of Table 1.

2. The method of claim 1 wherein said determining step comprises determining the effect of said agent on expression of at least two genes selected from said gene-set.

3. The method of claim 1 wherein said determining step comprises determining the effect of said agent on expression of at least three genes selected from said gene-set.

4. The method of claim 1 wherein said determining step comprises determining the effect of said agent on expression in said mammalian cell or mammal of at least one gene selected from GenBank accession numbers Al176456 metallothionein, X01118 gamma-rANP atrial natriuretic peptide, X89225 L-like neutral amino acid transport protein, J02722 heme oxygenase heat shock protein 32, and M957003 intercellular calcium-binding protein.

5. The method of claim 1 wherein said determining step comprises determining the effect of said agent on expression in said mammalian cell or mammal of at least one gene selected from GenBank accession numbers Af016296 neuropilin, X52140 integrin alpha-1, Ai638989 unknown, M899106 cyclin D2, A1170776 GRB2 growth factor receptor bound protein, A1008852 unknown, AA892801 unknown, X71127 complement protein C1q beta chain, U14950 synapse-associated protein 97, M57276 leukocyte antigen, AB005743 fatty acid transporter, U68272 interferon gamma receptor, and D85183 SHPS-1.

6. The method of claim 1 wherein said mammalian cell or mammal is a rat cell or a rat.

7. A method of identifying an agent that has cardiotoxic effects comprising treating a mammalian heart cell or a mammal with an agent; and determining the effect of said agent on expression in said mammalian cell or mammal of at least one gene selected from the gene-set of Table 1.

8. The method of claim 7 wherein said determining step comprises determining the effect of said agent on expression of at least two genes selected from said gene-set.

9. The method of claim 7 wherein said determining step comprises determining the effect of said agent on expression of at least three genes selected from said gene-set.

10. The method of claim 7 wherein said determining step comprises determining the effect of said agent on expression in said mammalian cell or mammal of at least one gene selected from GenBank accession numbers Al176456 metallothionein, X01118 gamma-rANP atrial natriuretic peptide, X89225 L-like neutral amino acid transport protein, J02722 heme oxygenase heat shock protein 32, and M957003 intercellular calcium-binding protein.

11. The method of claim 7 wherein said determining step comprises determining the effect of said agent on expression in said mammalian cell or mammal of at least one gene selected from GenBank accession numbers Af016296 neuropilin, X52140 integrin alpha-1, Ai638989 unknown, M899106 cyclin D2, A1170776 GRB2 growth factor receptor bound protein, A1008852 unknown, AA892801 unknown, X71127 complement protein C1q beta chain, U14950 synapse-associated protein 97, M57276 leukocyte antigen, AB005743 fatty acid transporter, U68272 interferon gamma receptor, and D85183 SHPS-1.

12. The method of claim 11 wherein said mammalian cell or mammal is a rat cell or a rat.
